(19) United States
(12) Patent Application Publication
(10) Pub. No.: US 2023/0108632 A1
(43) Pub. Date: Apr. 6, 2023

(54) Germicidal Lighting Device

(71) Applicant: Aleddra Inc., Renton, WA (US)

(72) Inventors: Chia-Yiu Maa, Bellevue, WA (US); Li-Jyuan Luo, Taipei City (TW); Chun-Te Yu, Bellevue, WA (US)

(21) Appl. No.: 17/495,031

(22) Filed: Oct. 6, 2021

(51) Int. Cl. A61L 2/00; G02B 5/20; F21V 8/00

(52) U.S. Cl. CPC A61L 2/0047; G02B 5/208; G02B 6/0026; G02B 6/0053; G02B 6/0055; G02B 6/0065; A61L 2202/11

(57) ABSTRACT

A germicidal lighting device includes an ultraviolet (UV) light source, a planar waveguide with a first surface, a second surface opposite to the first surface, and at least one edge surface perpendicular to the two parallel surfaces, and an optical filter. The UV light source is positioned adjacent to the edge surface of the planar waveguide to shine its UV light into the planar waveguide through the edge surface, and the UV light travels in the planar waveguide via total internal reflection. The first surface of the planer waveguide has a mechanism to reflect the UV light at an exit angle to exit the planar waveguide through the second surface. The optical filter filters a portion of the UV. Moreover, the light emitting area of the second surface of the planar waveguide is bigger than the light emitting surface area of the UV light source.

### BACKGROUND

### Technical Field

The present disclosure pertains to germicidal lighting devices and, more specially, proposes a germicidal light device using a waveguide.

### Description of Related Art

Germicidal lighting refers to the use of a light source emitting primarily ultraviolet (UV) wavelength in a range of 190 nm˜420 nm (with a peak performance at 254 nm) for disinfecting against bacteria and viruses in the air or on a surface. Germicidal lighting applications are not new. There is, however, a renewed interest of the germicidal lighting technologies and applications due to the COVID-19 pandemic. It is shown that a UVC dosage of 5 mJ/cm2 can disinfect against the SARS-CoV-II virus (the COVID-19 virus) with a 99.99% kill rate. This gives rise to the proliferation of germicidal lighting devices on the market. It is also known that over exposure of UV light can cause skin and eye damages to users.

Recent studies have demonstrated that a far-UVC light source emitting a light with a wavelength range 200˜230 nm has the effect of killing bacteria and viruses, yet without the side effect of causing skin and eye damages to a user. One such study can be found at https://www.cuimc.columbia.edu/news/far-uvc-light-safely-kills-airborne-coronaviruses. This leads to the possibility of using a far-UVC light source with a 200 nm˜230 nm wavelength range in germicidal lighting equipment. However, it is still possible to over-dose a user with far-UVC. American Conference of Governmental Industrial Hygienists (ACGIH) has published a UV Safety Guidelines as shown in FIG. 1 (ACGIH ISBN: 0-9367-12-99-6). It shows the UV Threshold Limit Values (TLVs), which is the maximum allowable dosage (in mJ/cm²) for each wavelength over an 8-hour period. It can be seen from FIG. 1, the TLV for 222 nm wavelength is set to 22 mJ/cm². Therefore, even with a 222 nm germicidal light source, it is not recommended to administrate more 22 mJ/cm² for an 8-hour workday.

The present disclosure proposes a germicidal lighting device that pairs a UVC light source with a planar waveguide to provide two advantages over the traditional germicidal lighting equipment: firstly, it reduces the UVC irradiation, and secondly, it widens the UVC emitting surface area. The combined result is that this proposed device can irradiate continuously at a lower UVC dosage to a larger area, thus providing a sustainable disinfection protection to the occupants in the space without exceeding the ACGIH TLV's.

### SUMMARY

In one aspect the germicidal lighting device includes an ultraviolet (UV) light source emitting a light in a 200˜400 nm wavelength range, a planar waveguide, and an optical filter. The planar waveguide has a first surface, a second surface opposite to the first surface, and at least one edge surface perpendicular to the first and the second surfaces. The UV light source is positioned adjacent to the edge surface of the planar waveguide to shine its UV light into the planar waveguide through the edge surface. The UV light travels in the planar waveguide via total internal reflection (TIR). The first surface of the planer waveguide comprises a mechanism to reflect the UV light at an exit angle to exit the planar waveguide through the second surface. The optical filter filters a portion of the UV light. Lastly, the light emitting area of the second surface of the planar waveguide is bigger than the light emitting surface area of the UV light source, thus having the effect of widening the UV light emitting surface as compared to that of the UV light source. Having the UV light traveling through total internal reflection in the waveguide, the irradiation of the UV light emitted out of the waveguide is reduced as compared to the UV light emitted by the UV light source.

In some embodiments, the optical filter is made of a lowpass optical filter with a cutoff wavelength in the 225˜235 nm wavelength range. For a far-UVC light source, even it has a peak wavelength around the 222 nm wavelength, there may still be wavelength greater than the 225 nm wavelength, which is harmful to the skin and the eyes of occupants in the space. Therefore, it is critical to use a lowpass optical filter for removing the wavelength above the 225˜235 nm wavelength range.

In some embodiments, the optical filter is positioned between the UV light source and the edge surface of the planar waveguide to filter the UV light before it enters the planar waveguide. In some other embodiments, the optical filter is positioned over the second surface of the planar waveguide, directly or indirectly, to filter the UV light after it exits the second surface of the planar waveguide.

In some embodiments, the mechanism for reflecting the UV light on the first surface at an exit angle to exit the planar waveguide through the second surface is accomplished through an etched pattern on the first surface. The etched pattern may be created via V-cutting, and often in both vertically and horizontally etched lines on the first surface.

In some embodiments, the present disclosure also has a first optical reflector surrounding the UV light source to redirect the UV light emitted out of the UV light source through the edge surface of the planar waveguide. The first optical reflector is useful when using with an omnidirectional UV light source. Without pairing a reflector to redirect the UV light of the omnidirectional UV light source, half of the UV light would not enter into the planar waveguide and thus be wasted.

In some embodiments, the present disclosure also has a second optical reflector on the first surface of the planar waveguide in order to redirect any portion of the UV light exit out of the first surface back through the planar waveguide, so that none of the UV light would leak out through the first surface. In some embodiments, the second optical reflector is implemented via a reflective coating on the first surface.

When the UV light exits out of the second surface of the planar waveguide, it may be at an angle not perpendicular to the second surface. It is more desirable to have the UV light shining in a direction perpendicular to the second surface in order to maximize the irradiation directly facing the second surface. Therefore, in some embodiments, the present disclosure has a brightness enhancement film (BEF) over the second surface of the planar waveguide to adjust the exit angle of the UV light off the second surface to result in the exit angle being more perpendicular to the second surface. It is common to use a double-layer BEF where one BEF layer corresponds to the UV light reflected by the vertical etched lines on the first surface whereas the other BEF layer corresponds to the UV light reflected by the horizontal etched lines on the first surface.

In some embodiments, the planar waveguide is made of quartz for the quartz material is known to have a good transmittance for the UV light. However, quartz-based waveguide can be expensive for making a larger planar waveguide. Another material, cyclic block copolymer (CBC) has demonstrated a sufficient transmittance to UV light, and thus may be considered as an alternative material for making the UV waveguide. Therefore, in some embodiments, the planar waveguide is made of CBC.

### BRIEF DESCRIPTION OF THE DRAWINGS

The accompanying drawings are included to aid further understanding of the present disclosure, and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 The Threshold Limit Values (dosage) according to ACGIH UV Safety Guidelines.

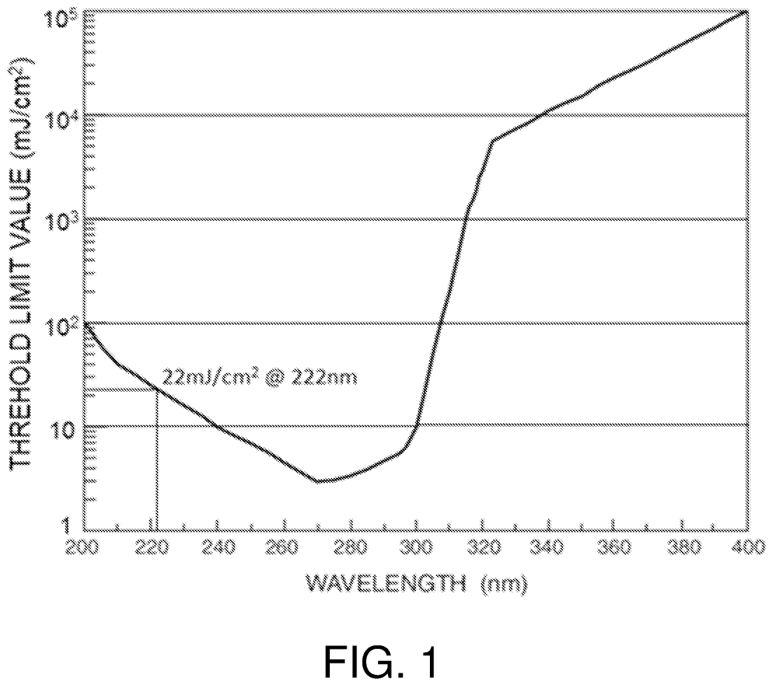

FIG. 2 schematically depicts a diagram of the present disclosure where the optical filter is located between the UV light source and the planar waveguide.

FIG. 3 schematically depicts another diagram of the present disclosure where the optical filter is positioned over the second surface of the planar waveguide.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of germicidal lighting devices having different form factors.

The present disclosure includes an ultraviolet (UV) light source, a planar waveguide with a first surface, a second surface opposite to the first surface, and at least one edge surface perpendicular to the two parallel surfaces, and an optical filter. The UV light source is positioned adjacent to the edge surface of the planar waveguide to shine its UV light into the planar waveguide through the edge surface, and the UV light travels in the planar waveguide via total internal reflection. The first surface of the planer waveguide has a mechanism to reflect the UV light at an exit angle to exit the planar waveguide through the second surface. The optical filter filters a portion of the UV. Moreover, the light emitting area of the second surface of the planar waveguide is bigger than the light emitting surface area of the UV light source. When using a far-UVC light source, the optical filter may be a lowpass filter with a cutoff wavelength in the 225˜235 nm wavelength range for removing the harmful UV wavelengths.

### Example Implementations

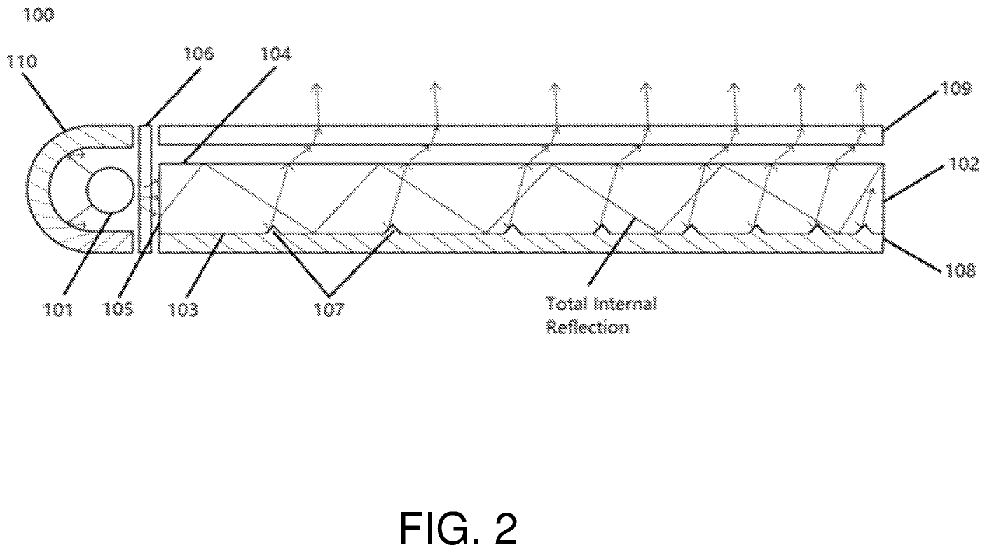

FIG. 2 is an embodiment of the germicidal lighting device of the present disclosure 100. The device 100 includes an omnidirectional UV light source 101, a planar waveguide 102, and an optical filter 106. The planar waveguide 102 has a first surface 103, a second surface 104, and an edge surface 105 which is perpendicular to the first surface 103 and the second surface 104. The reflector 110 directs the UV light emitted by the UV light source 101 toward the planar waveguide 102. An optical filter 106 is placed between the UV light source 101 and the edge surface 105 of the planar waveguide. The filter 106 is a lowpass filter with a cutoff wavelength at 230 nm. It blocks the wavelength above 230 nm and only permits the UV light less than 230 nm wavelength to pass through. The filtered UV light enters the planar waveguide 102 through the edge surface 105, and then travel within the planar waveguide via total internal reflection. A reflective coating 108 is used to improve the total internal reflection of the planar waveguide. The first surface 103 has an etched pattern 107 made of vertical and horizontal lines (though not shown) created by V-cutting. The UV light reflects off the etched pattern 107 at an exit angle that can exit the planar waveguide 102 through the second surface 104. The etched V-cuts 107 are more sparsely spaced when they are closer to the UV light source and become more densely spaced when they are away from the UV light source. This spacing pattern of the V-cuts helps producing a more uniformly distributed UV light out of the second surface 104. The UV light exiting out of the second surface 104 is at an exit angle not directly perpendicular to the second surface. A BEF 109 is used to adjust the exit angle of the UV light off the second surface 104 to result in the exit angle being more perpendicular to the second surface. The BEF 109 has two layers (though now shown) where one BEF layer corresponds to the UV light reflected by the vertical etched lines 107 on the first surface 103 and the other BEF layer corresponds to the UV light reflected by the horizontal etched lines on the first surface. The light emitting area of the second surface 104 of the planar waveguide is bigger than the light emitting surface area of the UV light source 101.

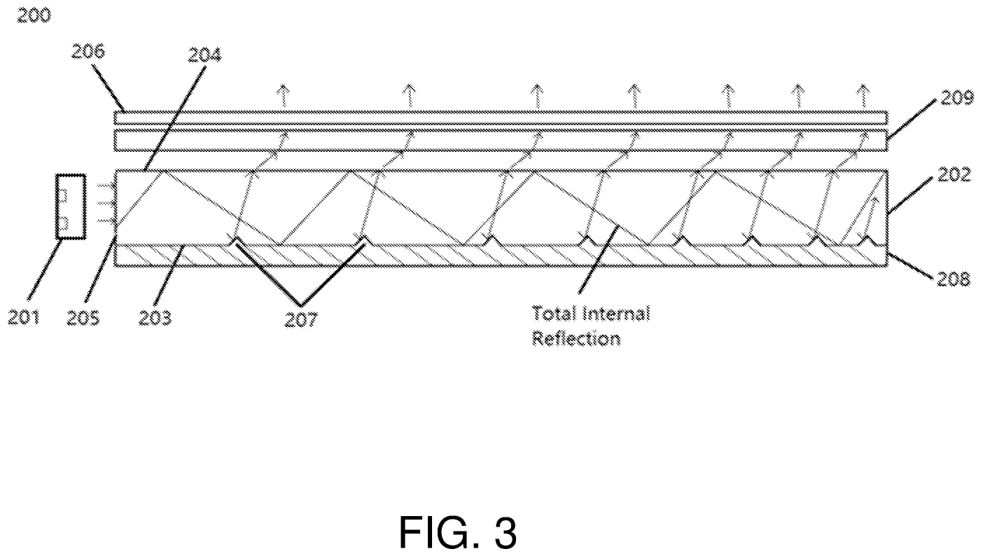

FIG. 3 is another embodiment of the present disclosure. This embodiment 200 is similar to the embodiment 100 in FIG. 2 with two differences. Firstly, the embodiment 200 uses a directional UV light source 201 with its light emitting surface facing the edge surface 205 of the planar waveguide 202. As such, it is not necessary to use a reflector to direct the UV light of the light source 201 toward the edge surface 205. Secondly, instead of placing a filter between the UV light source 201 and the edge surface 205 of the planar waveguide, a filter 206 is positioned over the second surface 204 of the planar waveguide 202 to filter indirectly the UV light after it exits the second surface and passes through the BEF 209.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques. As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A germicidal lighting device, comprising
an ultraviolet (UV) light source configured to emit a UV light in a wavelength range of 200˜400 nm;
a planar waveguide with a first surface, a second surface opposite to the first surface, and at least one edge surface perpendicular to the first and the second surfaces; and
an optical filter,
wherein:
the UV light source is positioned adjacent to the edge surface of the planar waveguide to shine the UV light into the planar waveguide through the edge surface,
the UV light travels in the planar waveguide via total internal reflection,
the first surface of the planer waveguide comprises a mechanism configured to reflect the UV light at an exit angle to exit the planar waveguide through the second surface,
the optical filter filters a portion of the UV light, and
a light emitting area of the second surface of the planar waveguide is bigger than a light emitting surface area of the UV light source.

2. The lighting device of claim 1, wherein the optical filter comprises a lowpass optical filter with a cutoff wavelength in a wavelength range of 225˜235 nm.

3. The lighting device of claim 2, wherein the optical filter is positioned between the UV light source and the edge surface of the planar waveguide to filter the UV light before the UV light enters the planar waveguide.

4. The lighting device of claim 2, wherein the optical filter is positioned over the second surface of the planar waveguide, directly or indirectly, to filter the UV light after the UV light exits the second surface of the planar waveguide.

5. The lighting device of claim 1, wherein the mechanism configured to reflect the UV light on the first surface at the exit angle to exit the planar waveguide through the second surface comprises an etched pattern on the first surface.

6. The lighting device of claim 1, further comprising:
a first optical reflector surrounding the UV light source and configured to redirect the UV light emitted out of the UV light source through the edge surface of the planar waveguide.

7. The lighting device of claim 1, further comprising: exit
a second optical reflector on the first surface of the planar waveguide and configured to redirect any portion of the UV light exit out of the first surface back through the planar waveguide.

8. The lighting device of claim 7, wherein the second optical reflector comprises a reflective coating on the first surface.

9. The lighting device of claim 1, further comprising:
a brightness enhancement film (BEF) over the second surface of the planar waveguide and configured to adjust the exit angle of the UV light off the second surface to result in the exit angle being more perpendicular to the second surface.

10. The lighting device of claim 1, the planar waveguide is made of quartz.

11. The lighting device of claim 1, the planar waveguide is made of cyclic block copolymer (CBC).
